(19) United States
(12) Patent Application Publication
(10) Pub. No.: US 2004/0146562 A1
(43) Pub. Date: Jul. 29, 2004

(54) Depot formulations in the form of a suspension

(75) Inventor: Jaymin C. Shah, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/689,778

(22) Filed: Oct. 21, 2003

Related U.S. Application Data
(60) Provisional application No. 60/421,295, filed on Oct. 25, 2002.

(51) Int. Cl. A61K 9/14

(52) U.S. Cl. 424/486

(57) ABSTRACT

A pharmaceutical kit for preparing an injectable depot formulation of an aryl-heterocyclic pharmaceutical compound, such as ziprasidone, and a method of preparing said depot formulation is disclosed.

This application claims priority under 35 USC 119(e) of U.S. Provisional 60/421,295, filed Oct. 25, 2002.

### FIELD OF INVENTION

The invention pertains to injectable depot formulations for aryl-heterocyclic compounds, such as arylpiperazinyl-C₂ and -C₄ alkyleneheterocycle compounds, including ziprasidone; and methods for making same. The injectable depot formulations of the invention permit controlled release of the active aryl-heterocyclic substances over prolonged periods of time after administration to a patient via intramuscular (IM) injection, for example. In a particular aspect, the invention pertains to a pharmaceutical kit wherefrom a suspension of ziprasidone serviceable as an injectable depot formulation can be prepared.

### BACKGROUND OF THE INVENTION

Certain aryl-heterocyclic compounds are known to have psychotropic effects. Ziprasidone in particular is a chlorooxyindole class aryl-heterocyclic that is an a typical anti-psychotic agent often prescribed for the treatment of schizophrenia. A typical anti-psychotics such as ziprasidone offer distinct advantages over traditional anti-psychotic medications insofar as they are associated with lower incidences of side effects, such as extrapyramidal symptoms (EPS), and confer greater efficacy of treatment to patients who are otherwise not responsive to more traditional drug therapies. Certain illnesses, such as schizophrenia, can be particularly difficult to medicate inasmuch as they are considered to be heterogeneous diseases whereby not all patients react similarly to the same treatment regimen. Exacerbating this is the problem that commonly attends long term treatment of schizophrenia; namely, non-compliance by patients with their dosage schedules. Indeed, it is conventionally thought that substantial numbers of schizophrenic patients are not or only partially compliant with their medication. Poor compliance can cause relapse into the psychotic condition thereby negating whatever benefits were achieved through treatment in the first place.

Where patient compliance is an issue, resort is sometimes had to long acting dosage forms of the medication. That is, dosage forms where a single administration leads to a sustained release of the medication over an extended period of time. This, in turn, simplifies the dosage regimen that a patient need adhere to, thus reducing the opportunity for non-compliance as occurs with a more rigorous schedule. Among such dosage forms is the depot formulation, which can be administered in various ways including intramuscularly by injection. The depot dosage injection is specifically formulated to provide slow absorption of the drug from the site of administration, often keeping therapeutic levels of same in the patient's system for days or weeks at a time. But there are instances where the use of a depot form has not been available. For example, in current practice, ziprasidone is administered once or twice daily in the form of an immediate release (IR) capsule for acute and long term treatment of schizophrenia; or is administered in intramuscular immediate release injection form for acute control of agitation in schizophrenic patients.

Ziprasidone is poorly soluble. Indeed, for the intramuscular immediate release formulation aforesaid, even ziprasidone mesylate, which is generally soluble relative to other known ziprasidone salts, has to be solubilized further, presently with the use of cyclodextrins as described in U.S. Pat. No. 6,232,304 incorporated herein by reference, to render it efficacious.

In the case of ziprasidone, it has been found that its poor solubility, which suggests amenability to a depot formulation where the drug should not be too soluble (to avoid burst) and release must be prolonged, does not in fact provide adequate pharmacokinetic exposure when constituted as such in a depot formulation.

Consequently, there is a need for an injectable depot formulation for aryl-heterocyclic compounds, such as ziprasidone, which can provide drug delivery over a sustained period of time at concentrations efficacious for treatment of, e.g. schizophrenia, in mammals including humans. In particular, there is a need for a pharmaceutical kit that can be conveniently employed to prepare such a depot formulation.

### SUMMARY OF THE INVENTION

The invention is premised on the finding that the solubilized forms of aryl-heterocyclics typically associated with (or at levels even greater than) immediate release can be surprisingly fabricated into depot formulations. In one aspect, the present invention is directed to a pharmaceutical kit comprising an aryl-heterocyclic compound, such as ziprasidone; which can be solubilized or unsolubilized; and a constituting liquid vehicle comprised of a viscosity agent with the proviso that when said aryl-heterocyclic compound is unsolubilized, said aqueous liquid further comprises a solubilizer.

### DETAILED DESCRIPTION OF THE INVENTION

The pharmaceutical kit of the invention conveniently provides an injectable depot formulation having significantly higher solubility of the aryl-heterocyclic drug in the formulation. The inventive kit achieves this improved drug loading and delivery by using solubilizers cooperatively with viscosity agents to obtain the controlled release typifying a depot effect.

The invention is useful in treating psychotic illnesses such as schizophrenia in mammals, including humans in need of such treatment. The invention is also useful in treating disorders and conditions, the treatment of which is facilitated by ziprasidone administration. Thus, the present invention has application where ziprasidone use is indicated as, e.g., in U.S. Pat. Nos. 6,245,766; 6,245,765; 6,387,904; 5,312,925; 4,831,031; and European EP 0901789 published Mar. 17, 1999, all of which are incorporated herein by reference.

The drug compounds contemplated for use in the present invention are aryl-heterocyclics, preferably those that have pharamacologic activity, e.g. psychotropic effects. Without limitation, an embodiment of an aryl-heterocyclic compound subject to the practice of the present invention has the structure:

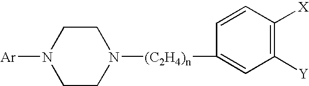

wherein

Ar is benzoisothiazolyl or an oxide or dioxide thereof, each optionally substituted by one fluoro, chloro, trifluoromethyl, methoxy, cyano, or nitro: n is 1 or 2; and

X and Y together with the phenyl to which they are attached form benzothiazolyl; 2-aminobenzothiazolyl; benzoisothiazolyl; indazolyl; 3-hydroxyindazolyl; indolyl; oxindolyl optionally substituted by one to three of (C₁-C₃) alkyl, or one of chloro, fluoro or phenyl, said phenyl optionally substituted by one chloro or fluoro; benzoxazolyl; 2-aminobenzoxazolyl; benzoxazolonyl; 2-aminobenxozazolinyl; benzothiazolonyl; benzoimidazolonyl; or benzotriazolyl. Representative examples of compounds falling within the foregoing definition are found in U.S. Pat. No. 4,831,031 incorporated herein by reference.

In one practice, the invention preferably applies to the above compounds wherein X and Y together with the phenyl to which they are attached form oxindole; more preferably, the oxindole moiety is 6-chlorooxindole-5-yl. In another preferred practice, Ar is benzoisothiazoyl; in still another preferred practice, n is 1. A particularly preferred aryl-heterocyclic to which the invention pertains is ziprasidone, 5-[2-[4-(1,2-benzisothiazol-3-yl)-1-piperazinyl]ethyl]-6-chloro-1,3-dihydro-2H-indol-2-one, which has the structure:

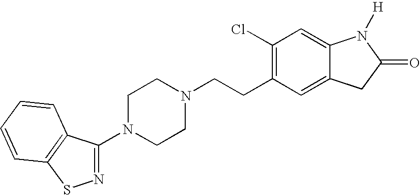

Although the aryl heterocyclic compound described herein may be constituted as a free base, it is preferred if aryl-heterocyclic compound is present as a pharmaceutically acceptable salt. The term “salt” in this regard intends pharmaceutically acceptable acid addition salts of aryl-heterocyclics, including ziprasidone. For purposes of preparing the kit or formulation of the invention, the salts can be anhydrous or in the form of one or more solvates, such as hydrates, including mixtures thereof. The salts may also occur in different polymorphic forms. By way of exemplification only, mesylate salts of the aryl heterocyclic ziprasidone may be present in dihydrate or trihydrate forms as disclosed in U.S. Pat. Nos. 6,110,918 and 6,245,765 both of which are incorporated herein by reference. Without limitation, preferred salts are selected from the group consisting of the tosylate, tartrate, napsylate, besylate, aspartate, esylate and mesylate salt. In an especially preferred practice, the aryl heterocyclic is ziprasidone mesylate, more preferably in the trihydrate form for purposes of making the kit or formulation. The term “ziprasidone”, as used herein, unless otherwise indicated, encompasses all such forms of ziprasidone, i.e. ziprasidone free-base, as well as all pharmaceutically acceptable salts of ziprasidone, including anhydrous and hydrated forms of such salts.

The pharmaceutical kit of the present invention provides an injectable depot formulation for delivery of the aryl heterocyclic active agent at concentrations effective for treatment of illnesses such as schizophrenia over a sustained period of time, i.e. for a period of time beyond that which is obtained by immediate release injection systems. Thus by way of further definition the injectable depot formulation of the present invention provides, for example, efficacious plasma levels of active agent for at least about 8 hours using typical injection volumes, e.g. about 0.1 ml to about 3 ml., about 1 ml to about 2 ml being usual. Preferably, the sustained period provided by the invention is at least about 24 hours; more preferably up to about 1 week; still more preferably from about 1 week to about 2 weeks or more including up to about 8 weeks using the injection volumes aforesaid. For example, in the case of ziprasidone, the practice of the invention can deliver at least about 0.5 to about 350 mgA/ml depot formulation. Thus, with an injection volume of about 1-2 ml, about 1 to about 700 mgA is delivered per injection over a sustained period of time. In another embodiment, from about 10 mgA to about 560 mgA ziprasidone is delivered over a sustained period of time. In further embodiments, from about 10 mgA (e.g. 5 mgA/ml) to about 420 mgA ziprasidone (e.g. 210 mgA/ml) is delivered per injection over a sustained period of time. In still a further embodiment, from about 10 mgA (e.g. 5 mgA/ml) to about 280 mgA (e.g. 140 mgA/ml) ziprasidone is delivered per injection for a sustained period of time. In another embodiment, from about 10 mgA to about 140 mgA (e.g. 70 mgA/ml) ziprasidone is delivered per injection over a sustained period of time. The preferred time period over which such amounts of ziprasidone are delivered by an injection are recited above, i.e. at least about 8 hours, preferably at least about 24 hours, more preferably at least about 1 week up to about 2 weeks, up to about 4 weeks and up to about 8 weeks also being preferred.

The pharmaceutical kit of the invention is comprised of at least two separate components: 1) a solubilized or unsolubilized aryl-heterocyclic compound, and 2) a liquid vehicle for constituting the aryl-heterocyclic compound into an injectable formulation. The liquid vehicle contains a viscosity agent, and when the aryl-heterocyclic is unsolubilized as herein defined, it further contains a solubilizer. When the two components of the kit are contacted, the solubilizer acts to solubilize the aryl-heterocyclic sufficient to attain a formulation providing the depot effect contemplated hereby. The two components can be part of a unitary structure, e.g. a dual chamber entity and the like; or more preferably they are provided in separate packages, such as vials and the like as known to the art. Thus for example, a first package, e.g. vial, contains the aryl-heterocyclic, and a second package, e.g. vial, contains the liquid vehicle with the viscosity agent and solubilizer, if needed. The packages are preferably configured to permit intermixing of the contents of one into the other. In a preferred practice, the vials are made of glass or resin and are clear or colored, e.g. amber. Glass is preferred with amber being further preferred for the aryl-heterocyclic compound. The two components comprising the inventive kit will now be further described.

In the practice of the inventive kit the aryl heterocyclic compound is either solubilized or unsolubilized. The term “solubilized” and related variations of same as used herein means that the heterocyclic has a solubility in water that is in excess of its free or salt forms to a degree sufficient to provide the prolonged (depot) duration of systemic exposure of active agent at the therapeutic levels envisoned by the invention. Without limitation, the heterocyclic can be “solubilized” using a cyclodextrin or other solubilizer to achieve the increased solubility contemplated herein. Thus the heterocyclic may be partly or fully solubilized and meet the definition of “solubilized.” Conversely, the term “unsolubilized” and related variations of same as used herein means the heterocyclic has a solubility that is in kind and/or degree insufficient to provide the aforesaid depot effect as contemplated. Under conditions where the aryl-heterocyclic is unsolubilized, the liquid vehicle comprising the viscosity agent further contains a solubilizer. In this practice, a sufficient amount of solubilizer is present in the liquid vehicle to solubilize enough of the unsolubilized heterocyclic to render it soluble for the depot purpose intended.

It will be understood that various embodiments of the present kit are available, and that all are within contemplation of the invention. For example, in one embodiment, the aryl-heterocyclic compound is sufficiently solubilized to provide the intended depot effect; in this circumstance, the liquid vehicle may, but need not, contain any additional solubilizer. The solubilized aryl-heterocyclic in this regard can be in the form of a pre-formed complex with a cyclodextrin as for example described herein. In another embodiment, the aryl-herterocyclic can be partly solubilized, but not enough to achieve the intended effect, i.e. the heterocyclic is “unsolubilized” for purposes of this specification. In this circumstance, the liquid vehicle contains at least sufficient solubilizer to make up the difference to solubilize enough of the remaining unsolubilized heterocyclic to provide the intended effect. Another embodiment is where the aryl-heterocyclic is substantially not solubilized at all, i.e. it is “unsolubilized” for purposes of this specification. In this instance, the liquid vehicle contains sufficient solubilizer to solubilize enough if not substantially all of the heterocyclic to obtain the depot effect. In practices where the aryl-heterocyclic is unsolubilized and the liquid vehicle contains the requisite solubilizer in type and amount, it is preferred that when the liquid vehicle is contacted with the aryl-heterocyclic compound, that contact occurs for a period of time sufficient to effect solubilization of the heterocyclic, prior to injecting the resultant depot formulation. For example, the two components should be allowed to contact for at least about 15 minutes, more preferably, between about 15 and about 45 minutes should elapse to effect solubilization prior to injection. As will be appreciated by those of skill in the art, this time can be shortened to less than 15 minutes by e.g. heating and/or the use of a sonicator, vortexor, mixer and the like. It is further preferred that just prior to injection, the constituted suspension is agitated, e.g. shaken, preferably for about 1 minute or more, e.g. about 2 minutes.

For convenience, the invention will now be further described exemplifying ziprasidone as the aryl heterocyclic compound. It is to be understood that the following discussion does not limit the scope of the invention and that the techniques hereinafter described appertain to and can be adapted for the family of aryl heterocyclics as disclosed herein. Other techniques that achieve the purposes stated can also be implemented and are envisioned as within the inventive practice.

The term “mgA/ml” as used herein relates to the weight (in mg) of aryl-heterocyclic compound, e.g. ziprasidone, per ml of composition to which the term is being applied. For ziprasidone free base, molecular weight=412.9.

In one embodiment, ziprasidone concentration is from about 0.5 mgA/ml to about 350 mgA/ml, for example at least about 60 mgA/ml, in the depot formulation of the present invention, which can include amounts in solution and amounts in suspension as appertain. More preferably for ziprasidone, concentration is between about 70 mgA/ml and about 280 mgA/ml depot formulation, including between about 140 mgA/ml and about 210 mgA/ml of depot formulation; higher concentrations are also within the scope of the inventive practice. Various techniques to solubilize ziprasidone to obtain these levels of concentration involve, non-limitingly, the use of cyclodextrins and other solubilizers.

The preferred solubilizer is a cyclodextrin. Cyclodextrins are cyclic oligosaccharides with hydroxyl groups on the outer surface and a void cavity in the center. The outer surface is usually hydrophilic hence cyclodextrins are soluble in water. The void on the other hand is typically hydrophobic. Cyclodextrins have the ability to form complexes with guest molecules, such as ziprasidone. Cyclodextrins contemplated by the invention include without limitation: α,β,γ-cyclodextrins, methylated cyclodextrins, hydroxypropyl-β-cyclodextrin (HPBCD), hydroxyethyl-β-cyclodextrin (HEBCD), branched cyclodextrins in which one or two glucoses or maltoses are enzymatically attached to the cyclodextrin ring, ethyl- and ethyl-carboxymethyl cyclodextrins, dihydropropyl cyclodextrins, and sulfoalkyl ether cyclodextrins, such as sulfobutyl ether-β-cyclodextrin (SBECD). The cyclodextrins can be unsubstituted or substituted in whole or in part as known in the art; mixtures of cyclodextrins are also useable. The preferred cyclodextrins for the depot formulation of the invention include γ-cyclodextrin, HPBCD, SBECD or mixtures thereof; SBECD being most preferred.

Cyclodextrin complexes with ziprasidone can be rendered soluble in water as described in U.S. Pat. No. 6,232,304 incorporated by reference above. For purposes of the invention, a pre-formed (solid) complex of cyclodextrin and ziprasidone can be employed as the first component of the inventive kit, or the cyclodextrin can be presented separately into the depot formulation to solubilize the ziprasidone, such as by adding the cyclodextrin in admixtrue with the viscosity agent or other components as part of the second component of the kit.

Viscosity agents used in the second component of the kit include those known in the art such as viscosified water, pharmaceutically acceptable oils and oil-based agents, polymeric agents and other non-aqueous viscous vehicles. Preferred viscosity agents include without limitation: cellulose derivatives, polyvinylpyrrolidone, alginates, chitosan, dextrans, gelatin, polyethylene glycols, polyoxyethylene ethers, polyoxypropylene ethers, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers and terpolymers of the foregoing, and mixtures thereof. Preferred cellulose derivatives include methyl cellulose, sodium carboxymethyl celluose (NaCMC) and hydroxypropyl methyl cellulose. Preferred polylactides, polyglycolides, copolymers and terploymers thereof include poly-lactic-co-glycolic acid (PLGA). Also contemplated as viscosity agents for the present invention are in situ gelling systems, e.g. stearic acid (SA) and N-methyl pyrrolidone (NMP) combinations, sucrose acetate isobutyrate and PLGA.

In a first solubilization embodiment, ziprasidone is solubilized with a cyclodextrin such as SBECD wherein the cyclodextrin is present in a concentration of up to about 60% w/v; more preferably, a concentration of about 40% w/v; still more preferably, a concentration of about 30%. In another embodiment, the depot formulation comprises a concentration of cyclodextrin, e.g. SBECD, of from about 5% to about 35%, especially from about 10% to about 20%. In a preferred aspect, the depot formulation in this regard takes the form of an aqueous suspension wherein the viscosity agent, e.g. NaCMC or the like, is present in water, e.g. sterilized water for injection, in an amount sufficient to render the viscosity of the depot formulation greater than 3.2 cps, preferably between about 20 cps to about 200 cps, more preferably, between about 30 cps to about 165 cps. For example, NaCMC can be present in an amount of from about 0.1% to about 3% w/v, preferably about 0.5% w/v to about 2% w/v. A pharmaceutically acceptable surfactant can optionally be used; surfactant in this regard can be present in an amount e.g. of up to about 1% w/v; preferably about 0.01 to about 0.1%; a preferred surfactant is a polyoxyethylene sorbitan ester, preferably Polysorbate 80 (Tween 80).

In a second solubilization embodiment, a complex of ziprasidone and a cyclodextrin is formed and isolated as a solid. This solubilized solid complex can then be suspended in a suitable viscosity vehicle, including non-aqueous viscous agents in which the ziprasidone-cyclodextrin complex is not soluble. Without limitation, a solid preformed complex can be obtained by lyophilizing the high concentration solution of the second embodiment described above. The lyophilized complex is suspended in non-aqueous viscosity agents including without limitation: sesame seed oil, including aluminum monostearate (ALMS) gelled sesame seed oil; and in situ gelling systems such as e.g. stearic acid (SA) and NMP combinations.

The liquid vehicle (second) component of the inventive kit can be aqueous or non-aqueous given choice of solubilization technique employed. In a preferred practice, the liquid vehicle is aqueous, e.g. comprises water for injection. The liquid vehicle contains one or more of the viscosity agents delineated above. In embodiments of the inventive kit where unsolubilized ziprasidone is employed as the first component, it is preferred if the liquid vehicle is aqueous and contains a cellulose-derived viscosity agent; it is further preferred in this instance that the liquid vehicle contain a cyclodextrin as a solubilizer. The amount of viscosity agent and solubilizer can vary depending, e.g. upon the dosing parameters described herein, although the final viscosity of the depot formulation from the kit must be greater than 3.2 cps, preferably between about 30 and about 165 cps.

In a preferred embodiment, the pharmaceutical kit comprises a first package containing ziprasidone powder in an amount sufficient to provide at least about 10 mg to about 30 mg per day of ziprasidone for at least about 8 hours, more preferably at least about 24 hours, even more preferably from about 1 to about 2 weeks, considering a usual injection volume of from about 1 ml to about 3 ml, preferably from about 1 ml to about 2 ml. The ziprasidone is preferably ziprasidone mesylate, more preferably ziprasidone mesylate trihydrate. In general, it is preferred if the aryl-heterocyclic compound is in a substantially dry form, e.g. a powder form, most especially a micronized powder form. It is further preferred if the contents of the first package are sterilized including without limitation sterilization by irradiation or e-beam. Sterilization by gamma or e-beam irradiation is preferred; most preferably, by gamma irradiation, even more preferably by gamma irradiation in doses of up to about 40 kGy, e.g. about 15 to about 35 kGy, about 25 kGy being preferred, especially for ziprasidone mesylate.

In a preferred embodiment of the invention, the second package contains an aqueous solution of a cyclodextrin in a concentration of up to about 60% w/v; a cellulose-derived viscosity agent in a concentration of from about 0.1% w/v to about 3% w/v, preferably from about 0.5% to about 3% w/v. A pharmaceutically acceptable surfactant can also be present, optionally, in the second package, e.g. in a concentration of up to about 1% w/v. It is preferred that when the aryl-heterocyclic is ziprasidone, the viscosity agent is NaCMC, preferably in a concentration of about 0.1% to about 3%, preferably from about 0.5% w/v to about 2% w/v. The liquid vehicle is aqueous, preferably sterilized water for injection. The solubilizer preferably is SBECD, present in a concentration of from about 5% w/v to about 35% w/v of said water; and the optional surfactant is present and is, without limitation, preferably a polyoxyethylene sorbitan ester such as e.g. Polysorbate 80, Tween 80; more preferably the surfactant is present in a concentration of about 0.01 to about 0.1% w/v. The water for injection is preferably present in an amount to provide an injection volume of about 1 to about 3 ml per injection. It is preferred that the second package and its contents be sterilized by suitable means, e.g. steam (autoclaving) sterilization at about 121° C. for about 15 minutes.

In one embodiment, the pharmaceutical kit of the invention is comprised of a first vial of (unsolubilized) ziprasidone mesylate trihydrate as a sterilized, micronized powder, preferably in an amount of about 239 mg (equivalent to about 175 mgA of ziprasidone); and a second vial of an aqueous vehicle comprising sterilized water for injection, SBECD at about 30% w/v, about 0.5% NaCMC w/v, and about 0.02% Polysorbate 80 (Tween 80); total volume of the aqueous vehicle so comprised in the second vial is about 3 ml. The pharmaceutical kit of this practice can be deployed to prepare 2.5 ml of 70 mgA/ml ziprasidone aqueous suspension.

Various embodiments of the present invention wherein the kit is comprised of unsolubilized ziprasidone (Vial 1) and a solubilzer (SBECD) and optionally a surfactant (Tween 80) in water for injection wherefrom an aqueous suspension of 70 mgA/ml ziprasidone useful e.g. for intramuscular depot injection are provided in Table 1. Table 2 provides embodiments of the invention wherein the kit is for preparation of aqueous suspensions for, e.g. intramuscular injection, comprising 140 mgA/ml ziprasidone and 210 mgA/ml ziprasidone.

TABLE 1
Various combinations of vials and dosing instructions to prepare
70 mgA/ml Aqueous Suspension for IM Depot Injection:

| Formulation No. | Vial 1: Drug Powder | Vial 2: Vehicle | Dosing Instruction |
|---|---|---|---|
| 1 | Ziprasidone | 30% SBECD + 0.5% | Constitute and dose |
| | mesylate | NaCMC | immediately |
| 2 | Milled drug | 30% SBECD + 0.5% | Constitute and dose |
| | | NaCMC | immediately |
| 3 | 1:1 ratio of | 0.5% NaCMC and | Constitute and dose |
| | drug:complex | 0.1% Tween 80 | immediately |
| 4 | Ziprasidone | 30% SBECD + 0.5% | Dose after incubation at |
| | mesylate | NaCMC + 0.1% | 50° C. for 1 hr |
| | | Tween 80 | |
| 5 | Ziprasidone | 30% SBECD + 0.1% | Constitute and dose |
| | mesylate | Tween 80 | immediately |
| 6 | Ziprasidone | 40% SBECD + 0.5% | Constitute and dose |
| | mesylate | NaCMC | immediately |
| 7 | Ziprasidone | 30% SBECD + 0.5% | Dose after incubation at |
| | mesylate | NaCMC + 0.1% | 50° C. for 1 hr |
| | | Tween 80 | |
| 8 | Ziprasidone | 30% SBECD + 0.1% | Dose after incubation at |
| | mesylate | NaCMC + 0.02% | 30° C. for 1 hr |
| | | Tween 80 | |
| 9 | Ziprasidone | 30% SBECD + 0.5% | Dose after incubation at |
| | mesylate | NaCMC + 0.02% | 30° C. for 1 hr |
| | | Tween 80 | |
| 10 | Ziprasidone | 30% SBECD + 0.25% | Dose after incubation at |
| | mesylate | NaCMC + 0.02% | 30° C. for 1 hr |
| | | Tween 80 | |

TABLE 2
Various combinations of the two vials and dosing instructions to
prepare 140 and 210 mgA/ml aqueous suspensions with
vehicle containing 10 and 20% SBECD:

| Formulation No. | Vial 1: Drug Powder | Vial 2: Vehicle | Dosing Instruction |
|---|---|---|---|
| 1 | Ziprasidone mesylate | 1.5% NaCMC 7LF, | Constitute and dose |
| 140 mgA/ml in | 735 mgA/vial | 10% SBECD, 0.1% | within 15 to 45 |
| vehicle with 10% | | Tween 80 | minutes |
| SBECD | | 4.6 ml | |
| 2 | Ziprasidone mesylate | 0.5% NaCMC 7H3SF, | Constitute and dose |
| 140 mgA/ml in | 735 mgA/vial | 20% SBECD, 0.1% | within 15 to 45 |
| vehicle with 20% | | Tween 80 | minutes |
| SBECD | | 4.6 ml | |
| 3 | Ziprasidone mesylate | 1.5% NaCMC 7LF, | Constitute and dose |
| 210 mgA/ml | 735 mgA/vial | 10% SBECD, 0.1% | within 15 to 45 |
| in vehicle | | Tween 80 | minutes |
| with 10% SBECD | | 2.9 ml | |
| 4 | Ziprasidone mesylate | 0.5% NaCMC 7H3SF, | Constitute and dose |
| 210 mgA/ml | 735 mgA/vial | 20% SBECD, 0.1% | within 15 to 45 |
| in vehicle | | Tween 80 | minutes |
| with 20% SBECD | | 2.9 ml | |

The following examples are illustrative only; they are not to be construed as limiting the scope or spirit of the invention.

### EXAMPLE 1

An embodiment of a pharmaceutical kit contemplated by the present invention is prepared as follows:

Vial-1: Into a 10 ml Amber Glass vial that was pre-washed, approximately 239 gms of ziprasidone mesylate trihydrate was manually added (equivalent to about 175 mgA per vila.) The vial was stoppered and crimped whereafter it was sterilized by gamma radiation at 25 kGy±10% dose. Vial-1 as constituted pursuant to the invention contained 239 mg of mesylate trihydrate equivalent to 175 mgA of ziprasidone.

Vial-2: An aqueous liquid comprising a viscosity agent and solubilizer was prepared as follows: approximately 15 mg of NaCMC 7H3SF was dispersed in approximately 1600 mg of water for injection at room temperature with stirring at 350 RPM for over 2 hours until complete dissolution and hydration of the NaCMC was achieved. Afterward, approximately 900 mg of SBECD was dissolved in the NaCMC solution while stirring. Polysorbate 80 in an amount of approximately 0.6 mg was added and make up water for injection was added to bring the total of water for injection used to about 2441.4 mg. The resultant solution was filtered through a filter train consisting of a 10 μm polypropylene filter and a 6 μm polypropylene filter. Initial filtrate was discarded and subsequent filtrate was collected. About 3 ml of this subsequent filtrate was added into a 10 ml Flint Type 1 Molded Glass vial. The vial was stoppered and sealed and then sterilized by autoclaving at about 121° C. for about 15 minutes. Vial-2 as constituted pursuant to the invention was an aqueous vehicle (3 ml) containing 30% w/v SBECD, 0.5% w/v NaCMC and 0.02% Polysorbate 80 (Tween 80).

### EXAMPLE 2

This example demonstrates the dissolution profile of ziprasidone in terms of concentration in solution, after constitution, over time. A first set of 15 pharmaceutical kits were made in accordance with Example 1. A second set of 15 pharmaceutical kits representing another embodiment of the invention were made using the same procedure as in Example 1 but for the fact that the viscosity agent was NaCMC 7LF instead of NaCMC 7H3SF. NaCMC 7LF has a lower viscosity than NaCMC 7H3SF.

Each kit was constituted into an injectable aqueous suspension depot formulation as follows: Approximately 2.3 ml of the aqueous vehicle from Vial-2 was injected into Vial-1 containing the ziprasidone powder. The dissolution profile was determined using the 15 kits aforesaid for each embodiment, at a protocol of 3 kits at 5 different time points—namely, initial, 15 min., 30 min., 60 min. and 24 hrs. At each time point the suspension from 3 kits was filtered through a 0.22 μm membrane filter to obtain a clear supernatant for analysis. The vials designated as “initial” time point vials were prepared for HPLC analysis immediately after constitution, one at a time. The resulting dissolution profiles are reported below in Table 3:

TABLE 3

| | 0.5% NaCMC 7LF | 0.5% NaCMC 7H3SF |
|---|---|---|
| | mean concentration ± | mean concentration ± |
| Time | SD (mgA/ml) | SD (mgA/ml) |
| Initial (n = 3) | 14.2 ± 1.19 | 18.1 ± 0.48 |
| 15 min. (n = 3) | 21.4 ± 0.14 | 20.5 ± 1.21 |
| 30 min. (n = 3) | 22.2 ± 0.17 | 21.5 ± 0.05 |
| 60 min. (n = 3) | 22.0 ± 0.36 | 21.6 ± 0.46 |
| 24 hrs. (n = 3) | 24.7 ± 0.48 | 23.8 ± 0.12 |

As seen from Table 3, the concentration plateaus at approximately 21 to 22 mgA/ml from 15 to 60 minutes; thereafter there is only a slight increase in solution concentration of ziprasidone, irrespective of the viscosity of the vehicle. Thus, high viscosity of the solution does not affect solubility of ziprasidone. Once constituted, the suspension depot formulation can be dosed from 15 to 60 minutes without any significant difference in the amount of drug in solution that a patient would receive. Because the ziprasidone concentration does not change significantly after 15 minutes, it is a preferred practice for this embodiment of the invention to employ an equilibrium period of about 15 to about 60 minutes, more preferably about 15 to about 45 minutes following constitution of the suspension prior to administration.

### EXAMPLE 3

This example demonstrates the dissolution profile of injectable ziprasidone aqueous suspension depot formulations according to the present invention having 140 mgA/ml and 210 mgA/ml.

Each kit (140 mgA/ml ziprasidone and 210 mgA/ml ziprasidone) was constituted into an injectable aqueous suspension depot formulation as follows: vials filled with 959 mg were constituted with 4.4 ml of vehicle to result in 5 ml of 140 mgA/ml suspension, and vials filled with 1438 mg were constituted with 4.2 ml of vehicle to result in 5 ml of 210 mgA/ml suspension. After the vehicle was added using a 5-cc syringe equipped with an 18G needle, each vial was shaken by hand for 2 minutes and set aside for a desired period of time. Prior to sample collection, the samples were shaken for an additional 2 minutes (except the initial). The samples were collected at initial, 15 minutes, 45 minutes, 3 h, 6 h, and 24 h time points. Two kits or pair of vials were used for each time point and formulation configuration. The samples were centrifuged at 5000 rpm for 5 minutes at 25° C. The supernatant was collected and filtered through 0.45 μm filter (vehicle with 10% SBECD) or first through 1 μm and then 0.45 μm (vehicle with 20% SBECD due to high viscosity). Clear supernatant was used to prepare the HPLC samples and analyzed for drug concentration in solution as solubility. As seen from the following results, solution concentrations of ziprasidone in formulation are significantly higher than solubility of ziprasidone mesylate.

TABLE 4
Dissolution profile upon constitution of 140 mgA/ml aqueous
suspension for IM depot injection:

| | Vehicle containing 10% | Vehicle containing 20% |
|---|---|---|
| | SBECD | SBECD |
| | 1.5% NaCMC 7LF, 10% | 0.5% NaCMC 7H3SF, |
| | SBECD, and 0.1% | 20% SBECD, and 0.1% |
| | Tween 80 in water | Tween 80 in water |
| | Average | Average |
| | Concentration ± SD | Concentration ± SD |
| Time | (mgA/ml) | (mgA/ml) |
| Initial (n = 2) | 8.11 ± 0.05 | 13.97 ± 0.47 |
| 15 minutes (n = 2) | 9.22 ± 0.06 | 17.68 ± 0.35 |
| 45 minutes (n = 2) | 9.24 ± 0.20 | 17.73 ± 0.18 |
| 3 hours (n = 2) | 8.89 ± 0.05 | 17.72 ± 0.24 |
| 6 hours (n = 2) | 9.18 ± 0.09 | 17.54 ± 0.35 |
| 24 hours (n = 2) | 9.53 ± 0.19 | 17.39 ± 0.37 |

NaCMC 7LF and NaCMC 7H3SF are the low and high viscosity grades of NaCMC.

TABLE 5
Dissolution profile upon constitution of 210 mgA/ml aqueous
suspension for IM depot injection.

| | Vehicle containing 10% | Vehicle containing 20% |
|---|---|---|
| | SBECD 1.5% | SBECD 0.5% |
| | NaCMC 7LF, 10% | NaCMC 7H3SF, 20% |
| | SBECD, and 0.1% | SBECD, and 0.1% |
| | Tween 80 in water | Tween 80 in water |
| | Average | Average |
| | Concentration ± SD | Concentration ± SD |
| Time | (mgA/ml) | (mgA/ml) |
| Initial (n = 2) | 8.52 ± 0.22 | 17.40 ± 0.14 |
| 15 minutes (n = 2) | 9.17 ± 0.14 | 18.07 ± 0.49 |
| 45 minutes (n = 2) | 9.17 ± 0.05 | 17.80 ± 0.56 |
| 3 hours (n = 2) | 8.94 ± 0.27 | 17.29 ± 0.40 |
| 6 hours (n = 2) | 9.23 ± 0.08 | 18.18 ± 0.17 |
| 24 hours (n = 2) | 9.14 ± 0.13 | 17.56 ± 0.38 |

NaCMC 7LF and NaCMC 7H3SF are the low and high viscosity grades of NaCMC.

### EXAMPLE 4

This example demonstrates the pharmacokinetic profile of the depot formulation obtained using the pharmaceutical kit prepared in accordance with Example 1. A kit of Example 1 was constituted by injecting about 2.3 ml of the aqueous vehicle of Vial-2 into Vial-1 to provide 2.5 ml of 70 mgA/ml ziprasidone aqueous suspension. After constitution, the vial was shaken for about 1 minute whereafter it was set aside for about 15 minutes, then shaken again for about 1 minute. Viscosity was between about 31 and 165 cps. A 22 gauge, 1-1.5 inch needle was loaded with 2 ml of the depot formulation thus constituted to provide a dose of about 140 mg ziprasidone.

The pharmacokinetic (PK) profile of the foregoing aqueous suspension depot formulation obtained from the kit of the invention was investigated in beagle dogs and compared to the following: Comparative Sample (1): an immediate release formulation comprised of solubilized ziprasidone, but no viscosity agent; and Comparative Sample (2): an aqueous suspension comprised of a viscosity agent (SBECD) and unsolubilized ziprasidone. The results were as follows: Comparative Sample (1) showed no depot effect, i.e. the serum concentration of ziprasidone was not quantifiable after 48 hrs; there was no sustained serum concentration. Comparative Sample (2) showed a ziprasidone serum concentration of 4.6±2.4 ng/ml (mean of 12-336 hrs). The present invention on the other hand showed a ziprasidone serum concentration of 12.9±3.7 ng/ml, which represented an increase in depot effect of approximately 280% over that of the next closest sample, Comparative Sample (2).

### EXAMPLE 5

Table 6 shows pharmacokinetic profiles of aqueous suspension depot formulations having 140 mgA/ml ziprasidone and 210 mgA/ml ziprasidone using pharmaceutical kits according to the present invention. In addition to ziprasidone, each formulation comprised 0.1% Tween 80; the formulations comprising 10% SBECD additionally comprised 1.5% NaCMC 7LF, and the formulations comprising 20% SBECD additionally comprised 0.5% NaCMC 7H3SF. These profiles were obtained from six groups (Groups A-F) of beagle dogs injected with the indicated depot formulations in a similar manner as described in Example 4. The results are ng ziprasidone/ml plasma:

TABLE 6

| | Group A: | Group B: | Group C: | Group D: | Group E: | Group F: |
|---|---|---|---|---|---|---|
| | 140 | 140 | 210 | 140 | 210 | 210 |
| | mgA/ml in | mgA/ml in | mgA/ml in | mgA/ml in | mgA/ml in | mgA/ml in |
| | vehicle | vehicle | vehicle | vehicle | vehicle | vehicle |
| | containing | containing | containing | containing | containing | containing |
| | 10% | 10% | 10% | 20% | 20% | 20% |
| | SBECD; 1 | SBECD; 2 | SBECD; 1 | SBECD; 1 | SBECD; 1 | SBECD; 2 |
| | ml injection | ml injection | ml injection | ml injection | ml injection | ml injection |
| Time | volume | volume | volume | volume | volume | volume |
| 1 week | 25.1 | 26.5 | 23.4 | 30.3 | 36.0 | 46.2 |
| (168 h) | | | | | | |
| 2 weeks | 40.8 | 75.2 | 23.8 | 22.3 | 33.8 | 58.0 |
| (336 h) | | | | | | |
| 3 weeks | 10.9 | 20.6 | 7.71 | 7.69 | 9.30 | 17.0 |
| (504 h) | | | | | | |

### EXAMPLE 6

This example demonstrates the preparation of a solubilized ziprasidone solid for use in an embodiment of the pharmaceutical kit of the invention. The solubilized ziprasidone in this instance is a pre-formed complex of ziprasidone and a cyclodextrin.

An isolated pre-formed complex of ziprasidone mesyiate trihydrate and the cyclodextrin SBECD was prepared as follows. The isolated ziprasidone-SBECD complex in solid form can be provided as a component of the pharmaceutical kit of the invention. In one embodiment of same, the other component of the kit contains a liquid vehicle in which said complex is not soluble thereby forming a non-aqueous suspension of solubilized ziprasidone when the kit is constituted into a depot formulation.

A 1095.3 gm batch of solution was prepared in an 80° C. water bath. After SBECD was dissolved in sterilized water for injection (SWFI) ziprasidone mesylate trihydrate was added to the resulting solution. During the entire process, the solution was stirred magnetically. The drug solution (82 mgA/ml) was filtered through a 0.45 μm filter and 2 ml aliquots were pipetted into 20 ml vials.

The vials of solution prepared above were lyophilized to obtain the ziprasidone-SBECD complex as a freeze dried solid. A lyophilization cycle was used with the following conditions: 1) Freezing step: temperature was −55° C. at 1° C./minute; 2) Primary drying: from −55° C. to −32° C. at 0.05° C./minute, held at −32° C. for 7 days, vacuum 100 mTorr; 3) Secondary drying: from −32° C. to 8° C. at 0.1° C./minute, held at 8° C. for 20 hours, vacuum 70 mTorr, then from 8° C. to 30° C. at 0.1° C./minute, held at 30° C. for 20 hours, vacuum 70 mTorr. The complex was comprised of ziprasidone at approximately 80 mgA/ml with about 56% SBECD.

Samples of the lyophilized complex were suspended in the various biocompatible, sustained release non-aqueous vehicles. These formulations, and the ziprasidone serum concentrations that were achieved subsequent to their administration to beagle dogs, are show in Table 7 below:

TABLE 7

| | | Mean Serum |
|---|---|---|
| | | Concentrations (ng/ml) |
| No. | Depot Formulation | over 12-336 hours |
| 1 | Suspension in 2% Aluminum | 18 ng/ml |
| | Monostearate (ALMS) | |
| | gelled Sesame oil | |
| | (60 mgA/ml; 2 ml injection) | |
| 2 | Suspension in 100-300 mg | 18.76 ng/ml |
| | Stearic acid (SA) in NMP | |
| | (70 mgA/ml; 2 ml injection) | |

### EXAMPLE 7

This example demonstrates various representative dosing practices using an embodiment of the kit of the present invention. A kit made in accordance with Example 1 is provided. Constitution of same to create the injectable depot formulation is as follows:

A 3 ml Luer-Lok syringe equipped with a 22G 1 or 1.5 inch needle withdraws 2.5 ml of the liquid vehicle in Vial-2. Air bubbles are removed (e.g. by tapping). The volume of the liquid vehicle is brought to the 2.3 ml mark on the syringe. Vial-1 is agitated (e.g. tapped) to ensure the ziprasidone is at the bottom of the vial. The liquid vehicle in the syringe is injected into Vial-1, Vial-1 being in an upright position. Vial-1 is agitated again (tapped) to free any ziprasidone from the crease around the bottom of the vial. Before the syringe with the needle is removed from Vial-1, the plunger of the syringe is released to reduce the positive pressure build-up inside the vial. The syringe with the needle is removed without pressing the plunger. The resulting suspension is agitated (e.g. mixed, shaken) for 2 minutes. The vial is then set aside for 30±15 minutes. Immediately prior to dosing, the vial of thus constituted suspension is agitated (e.g. shaken) for 2 minutes. To dose, an appropriate syringe equipped with a 22G, 1 or 1.5 inch needle (or a 16-21 gauge needle) is used to withdraw an appropriate volume of the uniform suspension. Trapped air bubbles can be removed by tapping the barrel of the syringe. The volume of the suspension in the syringe is brought to the appropriate mark to deliver doses of 7 to 140 mgA as representatively described in Table 8.

TABLE 8

| Dose to be | | Dose | Actual Dose |
|---|---|---|---|
| delivered | | volume | delivered, mean ± |
| (mgA) | Syringe type | (ml) | SD (mgA) |
| 7 | 1-ml B-D Luer-Lok | 0.1 ml | 7.13 ± 0.21 |
| | syringe | | |
| 70 | 1-ml B-D Luer-Lok | 1 ml | 69.08 ± 1.05 |
| | syringe | | |
| 140 | 3-ml B-D Luer-Lok | 2 ml | 136.23 ± 2.39 |
| | syringe | | |

What is claimed is:

1. A pharmaceutical kit comprising:
(i) a solubilized or unsolubilized aryl-heterocyclic compound; and
(ii) a liquid vehicle comprising a viscosity agent, with the proviso that when said aryl-heterocyclic compound is unsolubilized, said liquid vehicle further contains a solubilizer.

2. The pharmaceutical kit of claim 1 wherein said aryl-heterocyclic compound is ziprasidone.

3. The pharmaceutical kit of claim 2 wherein said solubilizer is a cyclodextrin.

4. The pharmaceutical kit of claim 3 wherein said cyclodextrin is γ-cyclodextrin, β-cylcodextrin, HPBCD, SBECD, or a mixture thereof.

5. The pharmaceutical kit of claim 2 wherein said viscosity agent comprises a cellulose derivative, polyvinylpyrrolidone, alginates, chitosan, a dextran, gelatin, polyethylene glycols, polyoxyethylene ethers, polyoxypropylene ethers, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers or terpolymers of the foregoing, sucrose acetate isobutyrate, PLGA, stearic acid/NMP, or a combination thereof.

6. The pharmaceutical kit of claim 3 wherein said viscosity agent comprises a cellulose derivative, polyvinylpyrrolidone, alginates, chitosan, a dextran, gelatin, polyethylene glycols, polyoxyethylene ethers, polyoxypropylene ethers, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers or terpolymers of the foregoing, sucrose acetate isobutyrate, PLGA, stearic acid/NMP, or a combination thereof.

7. The pharmaceutical kit of claim 5 wherein said cellulose derivatives include methyl cellulose, sodium carboxymethyl cellulose or hydroxypropyl methyl cellulose; and wherein said polylactides, polyglycolides, or copolymers and terploymers thereof include poly-lactic-co-glycolic acid.

8. The pharmaceutical kit of claim 2 wherein said liquid vehicle (ii) further contains a pharmaceutically acceptable surfactant.

9. The pharmaceutical kit of claim 3 wherein said liquid vehicle (ii) further contains a pharmaceutically acceptable surfactant.

10. The pharmaceutical kit of claim 8 wherein said surfactant is a polyoxyethylene sorbitan ester.

11. A pharmaceutical kit for an injectable depot formulation comprising:
(i) a first package containing ziprasidone; and
(ii) a second package containing an aqueous solution of a cyclodextrin, a cellulose-derived viscosity agent, and optionally a pharmaceutically acceptable surfactant.

12. The pharmaceutical kit for an injectable depot formulation of claim 11 wherein said ziprasidone is present in an amount sufficient to provide at least about 0.5 to about 350 mg ziprasidone per ml; said cyclodextrin is present in an amount sufficient to form a concentration of up to about 60% w/v; said cellulose-derived viscosity agent is present in a concentration of from about 0.5 to about 3% w/v; and said surfactant is optionally present in an amount sufficient to form a concentration of up to about 1% w/v.

13. The pharmaceutical kit for an injectable depot formulation of claim 11 wherein said ziprasidone is present in an amount sufficient to provide at least about 10 mgA to about 210 mgA ziprasidone per ml of said depot formulation.

14. The pharmaceutical kit of claim 11 wherein said ziprasidone is ziprasidone mesylate; said cyclodextrin is SBECD; said cellulose-derived viscosity agent is NaCMC; and said optional surfactant is a polyoxyethylene sorbitan ester.

15. A pharmaceutical kit for preparing an intramuscular depot injection formulation of ziprasidone comprising:
(i) a first package containing sterilized, micronized ziprasidone mesylate; and
(ii) a second package containing a solution of: water suitable for injection; SBECD in an amount sufficient to form a concentration of about 5% to about 35% w/v of said depot injection formulation; NaCMC in an amount sufficient to form a concentration of about 0.1% to about 3% w/v of said depot injection formulation; and a polyoxyethylene sorbitan ester in an amount sufficient to form a concentration of up to about 1% w/v of said depot injection formulation;
wherein said solution of (ii) is present in an amount sufficient to provide an injection volume of about 1 to about 3 ml injection, and said ziprasidone of (i) is present in an amount effective to deliver about 10 to about 30 mg per day of ziprasidone for about 1 to about 2 weeks in said injection volume.

16. A method of preparing an injectable depot formulation comprising: contacting a solubilized or unsolubilized substantially dry aryl-heterocyclic compound with an aqueous liquid containing a viscosity agent and optionally a pharmaceutically acceptable surfactant to form a suspension, with the provisos that when said aryl-heterocyclic compound is unsolubilized: a) said aqueous liquid further contains a solubilizer, and b) said contacting is for a period of time sufficient to effect solubilization of said aryl-heterocyclic compound with said solubilizer prior to injecting said depot formulation.

17. The method of claim 16 wherein said aryl-heterocyclic compound is unsolubilized ziprasidone; said solubilizer is a cylcodextrin; and said viscosity agent is a cellulose derivative.
